(19) United States
(12) Patent Application Publication
(10) Pub. No.: US 2005/0197556 A1
(43) Pub. Date: Sep. 8, 2005

(54) Continuously adjustable neurofeedback device

(76) Inventor: Diane R. Stoler, Boxford, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 11/067,129

(22) Filed: Feb. 25, 2005

Related U.S. Application Data
(60) Provisional application No. 60/548,639, filed on Feb. 27, 2004.

(51) Int. Cl. A61B 5/04

(52) U.S. Cl. 600/383; 600/544

(57) ABSTRACT

An neurofeedback device for comfortably holding at least one electrode onto a patient's head is described. The device includes an electrode positioning device, such as a headband and one or more straps each connected at spaced points of the headband and extending over at least a portion of the patient's head. At least one electrode holder is carried by the electrode positioning device for holding an electrode in electrical contact with the patient's head. The neurofeedback device permits precise placement of electrodes on a patient's head regardless of the patient's head size and thus can be used for treatment, training, and/or diagnosis.

### RELATED APPLICATIONS

This application claims priority under 35 U.S.C. § 119(e) to U.S. Provisional Application Ser. No. 60/548,639, entitled “CONTINUOUSLY ADJUSTABLE NEUROFEEDBACK DEVICE,” filed on Feb. 27, 2004, which is herein incorporated by reference in its entirety.

### BACKGROUND OF INVENTION

1. Field of the Invention

This invention relates to a neurofeedback device used in electroencephalography.

2. Discussion of Related Art

Electrodes placed on the skull can be used to monitor brain activity or treat a patient. Up to 80 electrodes can be placed on a patient's head for a sleep study, medical evaluation (electroencephalogram (EEG)), quantitative EEG (Q-EEG), Mini-Q, and/or neurofeedback treatment. Current systems require the use of caps which have either embedded electrodes or fixed predefined positions at which to place an electrode. Furthermore, in order to provide sufficient electrical contact between the electrodes and the patient's scalp, a messy electro-paste or electro-gel is applied to the electrodes.

The predefined locations defined by current cap configurations are placed to conform to the International 10-20 System of electrode placement. As known to those of skill in the art, the International 10-20 System describes a standard positioning of 24 electrodes with respect to the underlying section of cerebral cortex. When desired, a subset of the 24 positions defined by the 10-20 System may be used. Additionally, if more electrodes are desired, a practitioner may interpolate additional electrodes between the locations described by the International 10-20 system. The International 10-20 System is described in greater detail in the Appendix to the present application which is hereby incorporated by reference as if recited in full herein. Because the International 10-20 System locates electrodes with respect to the underlying section of cerebral cortex, the distances between adjacent electrodes should ideally change between people with different head sizes and shapes. Therefore, when using caps with fixed interelectrode distances, the electrode position may be inaccurate and/or multiple cap sizes must be used.

### SUMMARY OF INVENTION

A neurofeedback device according to one aspect of the present invention provides adjustable electrode placement to permit precisely locating an electrode with respect to a patient's head.

According to another aspect of the present invention, a neurofeedback headband is configured to securely hold at least one electrode in place on a patient's head without causing significant discomfort to the patient. Due to the extended time period required for studies, comfort to a patient may be desirable, especially for patients whose scalps have become sensitive such as may result from traumatic brain injury or fibromyalgia.

According to yet another aspect of the present invention, the neurofeedback device provides extensive flexibility with respect to placement of an electrode holder, thus permitting any number of electrodes to be used on a single patient. This allows a single neurofeedback device to be used for training, assessment, and/or diagnosis. Therefore, one embodiment of the neurofeedback device may permit a user to have a single headpiece which can be used with any number of desired electrodes where the electrodes may be placed anywhere on a patient's head, thus permitting the single neurofeedback device to be used for both training and assessment.

According to still another aspect of the present invention, sufficient electrical contact between a patient's head and an electrode on the neurofeedback device can be obtained without the use of an electro-paste or electro-gel. Instead, a mixture of saline and baby shampoo may be used as an effective electrically conductive fluid. The use of baby shampoo and saline advantageously reduces the time required to set up and clean up (compared to electrically conductive pastes or gels).

One embodiment of the present invention comprises an electrode positioning device and an electrode holder sized and configured for holding an electrode, wherein the electrode holder is releasably attachable to the electrode positioning device.

Another embodiment of the present invention comprises a headband, a cross strap having first and second ends, wherein at least one of the first and second ends is releasably attachable to the headband, and a plurality of electrode holding elements releasably attachable to the cross strap and/or headband, wherein the placement of the plurality of electrode holding elements on the neurofeedback device is configured to be continuously adjustable.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a side view of the neurofeedback device according to one embodiment of the present invention;

FIG. 2 is a plan view of the outside surface of a headband placed flat according to one embodiment of the present invention;

FIG. 3 is a plan view of an inside surface of a headband placed flat according to another embodiment of the present invention;

FIG. 4 is a top view of the neurofeedback device shown in FIG. 1;

FIG. 5 is a top view of an alternate cross strap configuration for the neurofeedback device according to another embodiment of the present invention;

FIG. 6 is a rear view of an electrode holder according to one embodiment of the present invention;

FIG. 6A is a rear view of an alternate electrode holder according to another embodiment of the present invention;

FIG. 7 is a plan view of one embodiment of an inside surface of a cross strap according to one aspect of the present invention;

FIG. 8 is a plan view of an alternative embodiment of an inside surface of a cross strap according to one aspect of the present invention; and

FIG. 9 is a plan view of an inside surface of a cross strap according to yet another embodiment of the present invention.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

A neurofeedback device according to one aspect of the present invention includes a continuously adjustable configuration, such that any number of electrodes may be placed anywhere on the device. Thus, the single neurofeedback device may be able to fit a variety of head sizes snugly, without causing discomfort. Additionally, electrodes on the device may be placed virtually anywhere, such that regardless of a patient's head size or shape, the electrode may be precisely placed in any desirable location. Furthermore, a neurofeedback device configured as described herein may permit a practitioner to use a single device for training, assessment, and/or diagnosis. The neurofeedback device may permit sufficient electrical contact between the electrodes on the neurofeedback device and a patient without the use of an abrasive skin preparation gel for EEG and ECG such as Nùprep™ (a trademark of Weaver and Company of Aurora, Colo.), electropastes or electrogels.

The neurofeedback device may comprise an electrode positioning device, such as a cap, headband, or netting. The shape and configuration of the electrode positioning device is not important, as long as it substantially conforms to the shape of a patient's head. Additionally, the electrode positioning device is configured to removably hold at least one electrode in a position-adjustable manner. The electrode positioning device may thus be configured to attach to an electrode holder. The electrode holder may comprise a pocket, strap, fastener, or other means for attaching an electrode to the electrode placement device. The electrode holder may be capable of attaching to the electrode placement device in a plurality of positions, such that the placement of electrode(s) on the electrode placement device may be adjustable.

One embodiment of a neurofeedback device includes an electrode holder configured to hold at least one electrode, where the electrode holder may be removably attachable to an electrode positioning device such as a headband and/or a cross strap. The electrode holder may be configured as a pocket and may comprise an absorbent material such that at least a portion of the material comprising the electrode holder may at least partially retain an electrically conductive solution. The electrically conductive solution may therefore provide an electrically conductive pathway between the electrode and a patient's scalp when the electrode positioning device is placed on a patient's head.

Headband

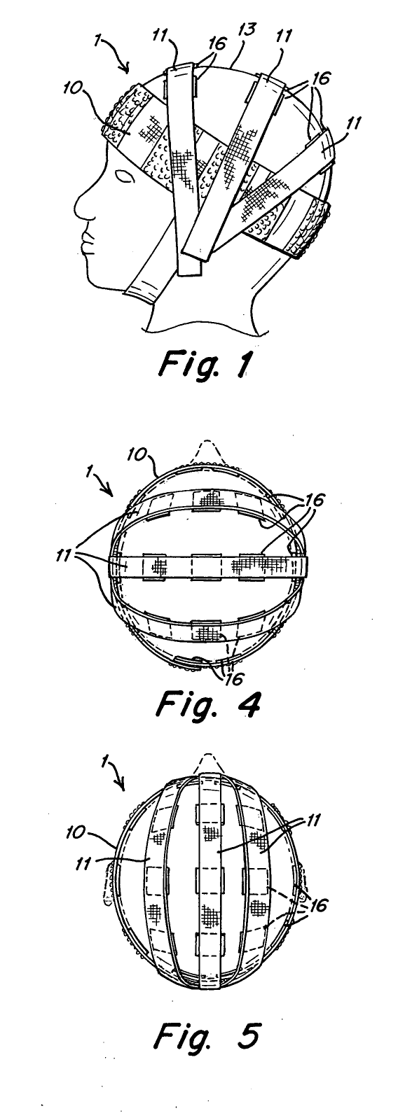

As shown in FIG. 1, the neurofeedback device 1 according to one embodiment of the present invention comprises an electrode positioning device. The electrode positioning device may comprise a headband 10, at least one cross strap 11, and an optional chin strap 12. In one embodiment, the headband 10 encircles the circumference of the head of a patient 13, and preferably is sized to fit snugly around a patient's head 13. Furthermore, the headband 10 preferably comprises a soft material, at least on the surface contacting the patient, in order to feel comfortable to the patient. Some examples of suitable materials for the headband 10 include a hook and loop strap (such as Velcro®), spandex, neoprene, elastic, rubber-like materials, belt webbing, cotton, terry cloth, and combinations thereof.

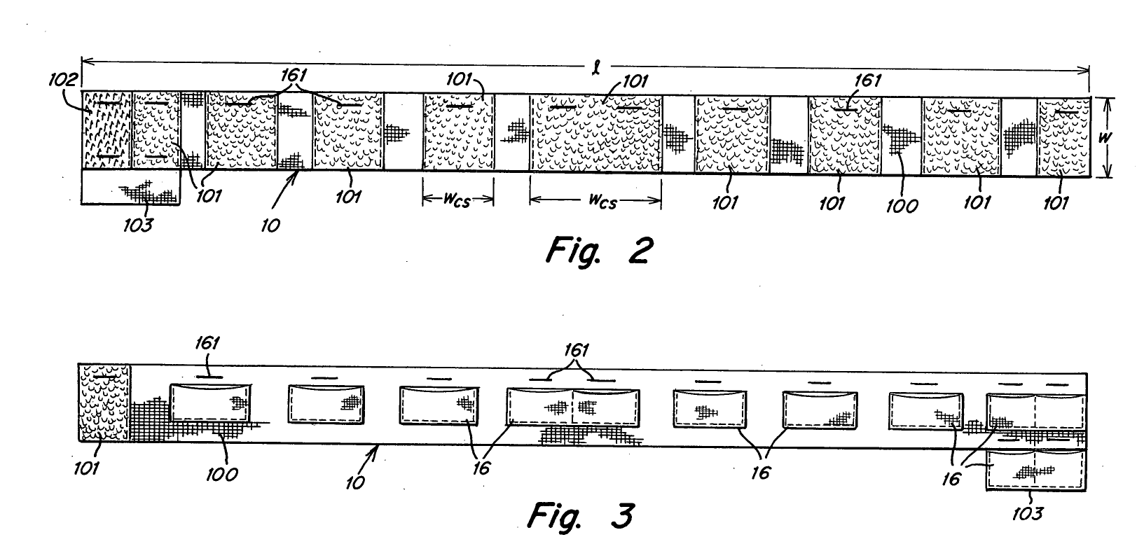

Additionally, in one embodiment, the headband 10 is adjustable. Thus, the headband 10 may be designed to fit a large range of head sizes. The headband 10 may be made from an elastic material and/or have a releasable fastener permitting a practitioner to selectively size the headband 10 to a desired size. The headband 10 may also be continuously adjustable. In other words, the headband 10 may not have discrete predefined fastening locations which permit a user to set the circumference of the headband 10 to a finite number of sizes. A single headband 10 could thus be used for children and adults by simply adjusting the circumference of the headband 10 or stretching the headband 10 to fit around a patient's head. In one embodiment and as shown in FIG. 2, the headband 10 can be comprised of a single strip, with releasably attachable means (such as a hook and loop fastener) to selectively create any desired headband circumference. For example, the headband 10 may be a strap between approximately 22 and 30 inches long (1) with releasably attachable fasteners at the ends. Preferably, the headband 10 may be 22.5, 24 or 30 inches long with one side of a hook and loop fastener attached to approximately the last 2 to 3 inches on each end. If hook and loop fasteners are used, the loops may be placed on the inside of the band 10 (i.e., the side contacting the head of the patient 13) with the hook portion on the outside in order to reduce discomfort to the patient.

In one embodiment, the headband 10 comprises both a resilient material and a fastener which permits continuous adjustability. For example, the headband 10 may comprise an elastic band 100 with at least one segment of a hook and loop fastener. As shown in FIGS. 2 and 3, at least one end of the headband may comprise a hook segment 102 of a hook and loop fastener, and the other end of the headband may comprise a loop segment 101 on the other side. In other words, as can be seen in FIG. 2, the outside surface of the headband 10 may comprise a hook segment 102 at one end. As can be seen in FIG. 3, the inside surface of the headband 10 may comprise a loop segment 101 at the other end. In this way, when the two ends of the headband 10 are placed together to form a circle, the hook segment 102 on the outside surface of the headband may contact the loop segment 101 on the inside surface of the headband 10. As will be apparent to one of skill in the art, the hook 102 and loop 101 segments may be reversed, or other fasteners may be used. By utilizing both an elastic material and a continuously adjustable fastener, the headband 10 may achieve both comfort and a precise fit. Other means of fastening a headband 10 in a continuously adjustable manner are within the scope of the present invention.

Fasteners located in the central portion of the headband 10 may be configured to retain at least one electrode and/or electrode holder. The fasteners may be releasable such that an electrode and/or electrode holder may be attached directly or indirectly to a fastener and later removed for cleaning or for adjustment to a different location. As shown in FIG. 2, the fasteners may comprise one part of a hook and loop fastener, such as the loop segments 101. The loop segments 101 in the central portion of the headband can be of varying widths (w_{cs}) as shown in FIG. 2. The loop segments 101 may thus cover the entire length of the headband 10, or only portions of the headband 10. The loop segments 101 thus permit a practitioner to place an electrode holder (and thus an electrode contained in the electrode holder) at a precise location with respect to a patient's head. As will be apparent to one of skill in the art, the hook segment or other fasteners could alternatively be used as the fastener on the headband 10 configured to attach an electrode and/or electrode holder.

The headband 10 may also comprise openings 161 as shown in FIG. 2. The openings 161 may be sized and configured to permit an electrode lead to be threaded through, but not large enough for an electrode to slide through. In this way, the openings 161 may provide anchoring means which prevent an electrode from slipping out of an electrode holder attached to the headband 10 when the neurofeedback device 1 is in use. Although the openings 161 are depicted horizontally through the loop section of the headband, the openings 161 may be vertically oriented or oriented at an angle, and may go through any part of the headband. Additionally, the openings may be linear, circular, polygonal, or any other shape, as long as they are sized and configured to permit passage of an electrode lead but not an electrode. Furthermore, the openings 161 may be defined at least in part by a small loop attached to an edge or face of the headband, sized such that it is sufficiently large to permit passage of an electrode lead but too small to permit passage of an electrode.

In another embodiment, the headband 10 comprises at least one electrode holder 16. The electrode holder 16 may be placed on the inside of the headband 10 (i.e., the surface which contacts a patient's head). The electrode holder(s) 16 may secure at least one electrode in a desired location with respect to a wearer's head. The electrode holder 16 may be a pocket sized and configured to hold at least one electrode. The electrode holder 16 may be configured to be slightly larger than an electrode, in order to firmly hold an electrode in a single location. However, as shown in FIG. 3, the headband 10 may alternatively or additionally comprise pockets 16 which may be large compared to the size of an electrode in order to permit precise placement of an electrode within a pocket. However, other means to attach an electrode to a desired location on the inner side of the headband 10 are also within the scope of the present invention.

As shown in FIG. 3, a headband which comprises electrode holders 16 permanently attached thereto may also comprise openings 161 sized and configured to permit passage of an electrode lead but not large enough to permit an electrode to slide through. As discussed above, the openings 161 may be of any shape or orientation. Furthermore, although the openings 161 are depicted in FIG. 3 as being above the electrode holder 16, the opening 161 may be on the side of the electrode holder 16, through an edge of the electrode holder 16, through a face of the electrode holder, below the electrode holder 16, or any other location. As described above, the openings 161 may also be defined at least in part by a loop which is located at an edge of the headband 10 and/or electrode holder 16.

A headband according to one aspect of the present invention may comprise an additional section or flap 103 extending perpendicular to the length of the band from one portion of the headband 10 as shown in FIGS. 2 and 3. The flap 103 may be comprised of any material, and may include means for attaching an electrode, such as an electrode holder or a fastener, or may even have at least one electrode permanently mounted there.

As shown in FIG. 3, the flap 103 may comprise at least one electrode holder 16 on the inside to permit an electrode to be placed therein. Preferably, the flap 103 comprises two electrode holders 16 which are sized and configured to each hold an electrode. In this arrangement, the electrode holders 16 may advantageously be used as a reference point to position the headband 10 on a patient's head. For example, the headband 10 may be placed on a patient's head such that electrodes in the electrode holders 16 on the flap 103 are on either side of a centerline on the back of a patient's head (corresponding to the O₁ and O₂ positions). The electrode holders 16 are preferably configured to permit electrical conduction through the electrode holder surface and provide a conductive path between the electrode and a patient's head. As described below, an electrode holder 16 does not need to be configured to substantially enclose an electrode. For example, a loop or plurality of loops which is configured to tightly hold an electrode would also be an electrode holder within the scope of the present invention. However, it is within the scope of the present invention for the electrode holder 16 to permanently enclose an electrode as well.

The width w of the headband 10 may be chosen so that the friction between the headband 10 and the user's head 13 is sufficient to hold the headband 10 securely in place without slipping off. For example, the headband may be between 1 inch and 3 inches wide. Preferably, the headband 10 is approximately 2 inches wide.

Any type of band may be used in accordance with one aspect of the present invention. In other words, in addition to the embodiments described above, any headband (e.g., an off-the-shelf headband of Terry cloth used as exercise attire) may be used in conjunction with the electrode holders as a portion of the neurofeedback device of the present invention.

Cross Straps

As shown in FIG. 1, in one embodiment of the present invention, the electrode positioning device also comprises at least one cross strap 11. The cross strap 11 is configured to hold at least one electrode holder 16 securely against the head of a patient 13. Cross straps 11 preferably are configured to reach from one point on the headband 10 across the top portion of the head of a patient to another point on the headband 10. These cross straps 11 are preferably configured to attach to the headband 10 such that the cross straps 11 can support at least one electrode holder 16 in a desired location. In one embodiment of the present invention, the cross straps 11 support at least one electrode holder 16 such that an electrode in the electrode holder 16 is placed in a location defined by the International 10-20 System. For example, as shown in FIGS. 1 and 4, there may be three cross straps 11, each extending from the headband 10 across the top portion of a patient's head 13 to another point on the headband 10. Although three cross straps 11 are shown, any number of cross straps 11 may be used. As shown in FIGS. 4 and 5, each of the cross straps 11 may be configured to support a plurality of electrode holders 16. Although FIGS. 4 and 5 show three cross straps, more or fewer may be used. For example, as few as one or as many as seven or more cross straps may be used.

As shown in FIGS. 1 and 4, the cross straps 11 may extend from side to side across the head of the patient. Alternatively, as shown in FIG. 5, the cross straps 11 may extend from the front to back of the patient's head. In both configurations, the cross straps 11 extend fully across the patient's head 13. However, as will be evident to one of skill in the art, the cross straps 11 may be configured to only extend partially across a patient's head 13 if there is at least one supporting element to attach to. For example, if there is a supporting strap which extends from the front to the back of the patient's head, a cross strap 11 may only extend from the headband 10 to the supporting strap.

In one embodiment, the cross straps 11 are adjustable. In other words, the cross straps 11 are detachable from the headband 10 on at least one end such that the length of the functioning portion of the cross strap 11 (i.e., the portion of the cross strap which may be used to position an electrode holder) is adjustable. Thus, the cross straps 11 may be releasably attachable to the headband 10 on at least one side via snaps, buttons, hooks, Velcro® (a registered trademark of Velcro Industries B.V.), or other releasably attachable means. The other end of the cross strap 11 may be permanently affixed to the headband 10, such as being sewn on, glued on, riveted on, or attached permanently by other means. Alternatively, both ends of the cross strap 11 may be releasably attachable to the headband 10.

Similar to the headband 10 configurations shown in FIGS. 2 and 3, the cross strap 11 may include at least one opening (not shown) which is sized and configured to permit passage of an electrode lead but not large enough to permit an electrode to fit through. As stated with respect to the openings in conjunction with the headband embodiments, the size, shape, location, and orientation of the openings in the cross strap are not limited by the locations, shapes, and orientations depicted in the Figures.

As shown in FIG. 7, in one embodiment the cross strap 11 comprises a band 110 with segments of a releasable fastener 111 on which to attach an electrode holder. The band 110 may be elastic, belt webbing, cotton, spandex, or any other resilient or non-resilient material. The releasable fastener may be one side of a hook and loop fastener. The combination of a resilient material 110 and sections with a releasable fastener 111 advantageously allow a user to utilize the resilient properties of the material while permitting releasable attachment of an electrode and/or electrode holder. In one embodiment, the cross strap 11 has three releasable fasteners 111 which are centered horizontally and vertically on the cross strap 11. More or fewer releasable fasteners 111 may also be used and are within the scope of the present invention. The releasable fasteners may be approximately 1.5 inches wide (wₕ) and spaced approximately between 0.5 and 1 inch apart (Sₕ). Although the releasable fasteners are depicted as taking up the entire width of the cross strap 11, the releasable fastener 111 may be wider or narrower than the width of the cross strap 11.

Each end of the cross strap 11 may comprise a segment of one side of a hook and loop fastener which may be configured to attach to a segment of the headband 10. In one embodiment of the present invention, the last approximately 2 inches on both ends of the cross strap 11 includes a segment of hook fastener as shown in FIG. 7.

In another embodiment, the electrode holders 16 are releasably attachable to any part of the cross strap 11. For example, as shown in FIG. 8, the entire central portion of the cross straps 11 may comprise the loop side of a hook and loop fastener and the electrode holders 16 may comprise the hook side of the hook and loop fastener. In this way, the neurofeedback device 1 permits continuous adjustment of the electrode holders 16 to permit precise placement of the electrodes on a patient's head.

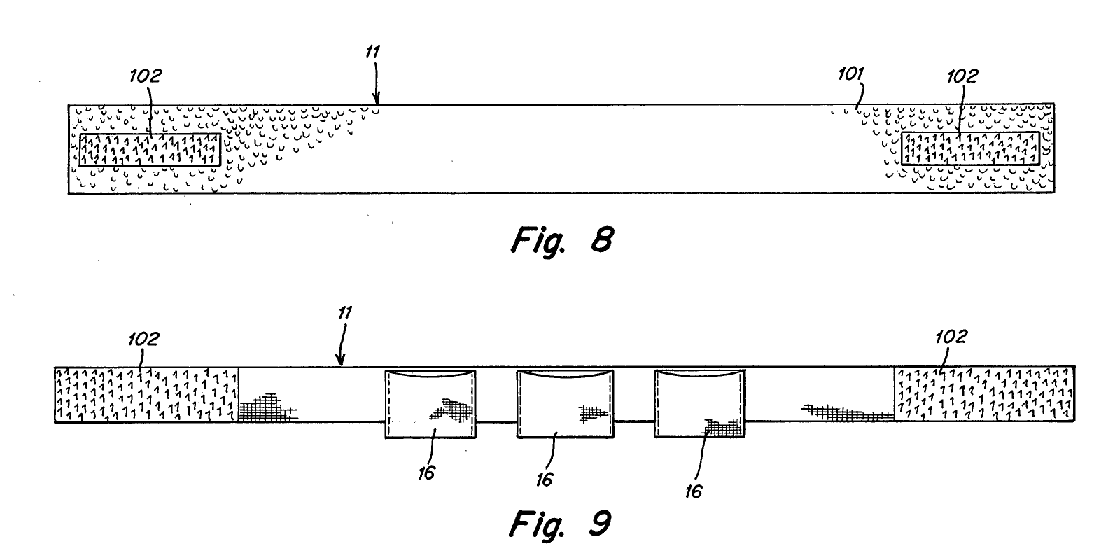

In another embodiment of the present invention and as shown in FIG. 9, at least one of the electrode holders 16 may alternatively or additionally be permanently attached to the cross strap 11. The electrode holders 16 may be sized to snugly receive the electrodes. Alternatively the cross straps 11 may have at least one fixed electrode holder 16 which is large compared to the size of an electrode in order to permit continuously adjustable placement of at least one electrode within the electrode holder 16. If the placement of an electrode holder 16 is fixed, the electrode holder 16 may be sewn on, glued on, or otherwise permanently affixed as will be apparent to one of skill in the art. As will also be apparent to one of skill in the art, at least one electrode itself may be permanently affixed to the cross strap 11. For example, an electrode may be permanently attached to the center of a cross strap 11 in order to align the cross strap 11 with the centerline of a patient's head.

The cross straps 11 are preferably sized to fit any sized head. Therefore, the cross straps 11 may be approximately 15 inches long, with releasable fasteners at least on approximately the last 3 inches of either one or both ends. In one embodiment, the cross straps 11 are between 12 and 13.5 inches long, with releasable fasteners in the last approximately 2.75 inches at each end. Other means to adjust the length of the cross straps, such as D-rings and buckles, may be used.

In each of the cross strap 11 configurations above, the cross strap 11 may be sized and configured to be secured to the headband 10 and capable of being used without slipping off a patient's head. Preferably, the cross strap 11 is between 0.75 inches wide and 2 inches wide.

Electrode Holder

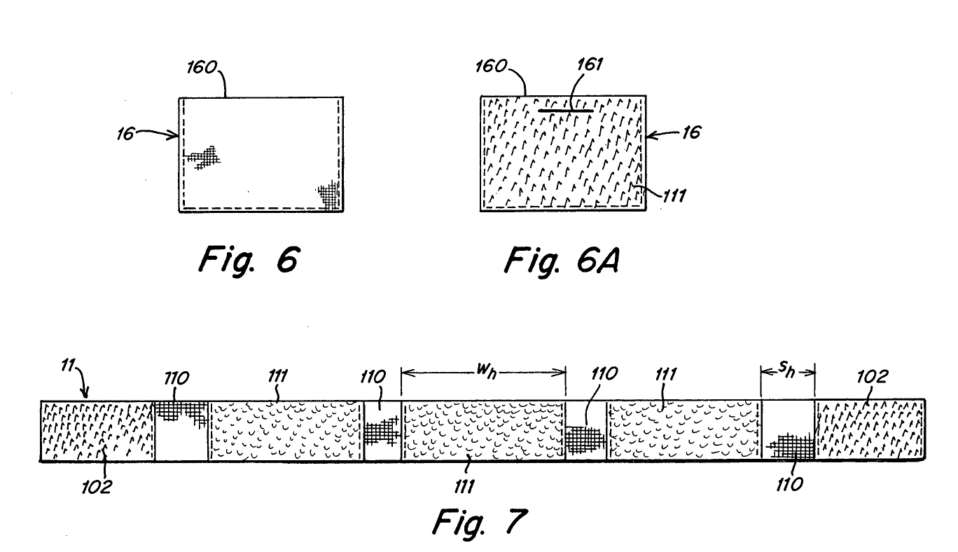

The neurofeedback device 1 according to one embodiment of the present invention additionally comprises at least one electrode holder 16 which is sized and configured to hold an electrode. In one embodiment, the electrode holder 16 comprises a pocket as shown in FIG. 6. Thus, the electrode holder 16 has an opening 160 on one side which enables an electrode to be placed therein. The electrode holder 16 may substantially surround an electrode placed therein. As will be apparent to one of skill in the art, the shape and configuration of the electrode holder 16 could be any number of shapes and configurations. Although the electrode holder 16 of FIGS. 6 and 6A are rectangular, the electrode holder could be circular, polygonal, or any other shape. The location of an opening 160 in the electrode holder 16 may also be in locations other than that depicted in FIG. 6. For example, an electrode holder 16 in the pocket configuration may have an opening 160 in a face such that the electrode holder 16 is slipped around the edge(s) of an electrode. Alternatively or additionally, an electrode holder 16 may be formed in a sleeve-like configuration and secured to the headband 10 and/or cross strap 11 with openings at opposing ends without departing from the scope of this invention. In one embodiment, the electrode holder 16 comprises at least one strap secured as by stitching or Velcro® to the headband 10 and/or cross strap 11 for retaining an electrode in place. The strap may include elastic or another resilient material in order to secure an electrode in place. Furthermore, a releasable fastener, such as a segment of hook and loop fastener, may also constitute at least a portion of the electrode holder 16. For example, one face of a pocket may comprise one side of a hook and loop fastener. Alternatively, a small segment of hook and loop fastener may be affixed to one side of an electrode to attach an electrode directly to the headband 10 and/or cross strap 11. In another embodiment of the invention, the electrode holder 16 is configured like a sock that fits over an electrode. Other configurations for electrode holders 16 also fall within the scope of the present invention and may be customized for different shaped electrodes.

In another embodiment of the present invention, the electrode holder 16 may comprise an opening 161 for an electrode lead. For example, as shown in FIG. 6A, the electrode holder 16 may comprise an opening 160 on one side and a second opening 161 through one face. The openings 160, 161 may be of different sizes. Therefore, for example, one opening 160 may be sized and configured to fit an electrode through. The second opening 161 may be substantially smaller such that an electrode lead may be threaded through, but the electrode is prevented from slipping through the second opening 161. In this embodiment, the electrodes may be prevented from falling out of the electrode holder 16. Although the openings 160, 161 are depicted such that one opening is on a face of an electrode holder 16 and the other on a side of the electrode holder 16, the openings 160, 161 may both be on sides of the electrode holder 16, both be on faces of the electrode holder 16, may both be on a single side of the electrode holder 16, or both be on a single face of the electrode holder 16. It is also within the scope of the present invention that at least one of the openings 160, 161 is in a corner of the electrode holder 16. Additionally, the openings 160, 161 may be on adjacent or non-adjacent sides and faces. Furthermore, at least one opening 161 may be defined by a loop attachment to an edge or face of the electrode holder 16. Alternatively or additionally, the electrode holder 16 may be designed to open in order to place an electrode in, and then be largely closeable such that the electrode is retained within the electrode holder 16 while the electrode lead extends from the electrode holder 16.

As mentioned above in conjunction with the openings in the headband 10 and cross strap 11, the opening 161 in an electrode holder 16 may be of any size, shape, and configuration. Although the opening 161 is depicted in FIG. 6A as being linear, the opening 161 may be circular, polygonal, or any other shape as long as it is sized and configured to permit passage of an electrode lead but not an electrode. Additionally, the opening 161 may be oriented horizontally as shown in FIG. 6A, or it may be oriented vertically or at any other angle.

The electrode holder 16 is preferably configured to permit electrical conduction from an electrode within the electrode holder 16 to a patient's head 13. In one embodiment, the electrode holder 16 comprises an absorbable surface on the side facing a patient's head, such that the absorbable surface absorbs and retains sufficient electrically conductive fluid to provide an electrical path between an electrode in the electrode holder 16 and a patient's head. Some examples of suitable absorbable materials include felt, gauze, chamois, and cotton. Alternatively or additionally, the electrode holder 16 may comprise an electrically conductive material. Additionally, the electrode holder 16 preferably comprises a soft material such that it cushions an electrode within the electrode holder 16 and provides a soft interface with the patient's head 13.

An electrode holder 16 not permanently attached to the cross strap 11 may be disposable. In this way, a practitioner would not need to purchase a new neurofeedback device 1 when the electrode holder 16 wears out. In one embodiment of the present invention, the electrode holder 16 is also washable in order to enable a practitioner to remove at least the residue of the electrically conductive fluid between patient uses.

Chin Strap

As shown in FIG. 1, the neurofeedback device 1 may also include a chin strap 12. The chin strap 12 may not be necessary for some patients. However, because the size and shape of patients' heads vary, it may be beneficial for at least some patients to secure a chin strap 12 onto the headband 10. The chin strap 12 may prevent the headband 10 from slipping upward, thus preventing interruption of the electrical contact between the electrodes on the neurofeedback device 1 and the patient's head 13. If a chin strap 12 is used, the chin strap 12 may include an elastic material in order to comfortably provide a snug fit. Additionally, the chin strap 12 is preferably removable from the headband 10 such that it is out of the way when not in use. The chin strap 12 may thus be attached to the headband 10 via releasable fasteners such as hook and loop fasteners, snaps, hooks, buttons, and other means known to those of skill in the art.

Method of Use

In use, a practitioner would determine where the electrode holders 16 should be placed with respect to a patient's head. The adjustable design of the neurofeedback device may permit a practitioner to place electrodes clustered in a very specific area of interest, or space them out according to the International 10-20 System, or any variation thereof. The practitioner would then place the electrode holders in the desired locations on the electrode positioning device, such as in the configuration of FIGS. 4 or 5. For example, the practitioner could place electrode holders in at least one of the positions indicated by FIG. 4 or 5. Electrode(s) could then be placed in the electrode holder(s). Alternatively, the practitioner could place the desired number of electrodes in electrode holders and then attach the electrode holders to the electrode positioning device in the desired positions. If the electrode holders and/or electrode positioning device is configured to have a separate opening for the electrode lead, the lead may have to be threaded through the opening configured for the electrode lead prior to placing the electrode in the associated electrode holder. If the electrode holders are configured to slip over the electrodes, then the electrode may be positioned on the electrode positioning device and the electrode holders subsequently slipped over the electrodes.

The practitioner would then apply a sufficient amount of electrically conductive fluid, such as baby shampoo and saline, to the surface of the electrode carrying means to create an electrically conductive path between an electrode in the electrode holder and a patient's head. Other electrically conductive fluids may be used in conjunction with the neurofeedback device of the present invention as well. Finally, the practitioner would place the neurofeedback device on the head of a patient by snugly fitting the electrode positioning device to the patient's head and adjusting any fastener(s) and electrode holder(s) as necessary.

Having thus described several aspects of at least one embodiment of a neurofeedback headband, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A neurofeedback device, comprising:
an electrode positioning device; and
an electrode holder sized and configured for holding an electrode, wherein the electrode holder is releasably attachable to the electrode positioning device.

2. The neurofeedback device according to claim 1, wherein the electrode positioning device comprises a cross strap or headband.

3. The neurofeedback device according to claim 1, wherein the position of the electrode holder on the electrode positioning device is adjustable.

4. The neurofeedback device according to claim 1, wherein at least a part of the electrode positioning device is adjustable.

5. The neurofeedback device according to claim 1, wherein the electrode positioning device comprises elastic or belt webbing.

6. The neurofeedback device according to claim 2, wherein the electrode positioning device comprises both a cross strap and a headband, wherein the cross strap is releasably connected to the headband.

7. The neurofeedback device according to claim 1, wherein the electrode holder is releasably connected to the electrode positioning device by a hook and loop fastener.

8. The neurofeedback device according to claim 1, wherein the electrode holder or electrode positioning device includes a opening sized and configured to permit an electrode lead to be threaded through.

9. The neurofeedback device according to claim 1, wherein the electrode holder comprises a pocket.

10. The neurofeedback device according to claim 1, wherein the electrode holder comprises a loop.

11. The neurofeedback device according to claim 1, wherein the electrode holder is substantially larger than an electrode to be placed therein, such that the electrode may be positioned in many locations within the electrode holder.

12. The neurofeedback device according to claim 1, wherein the electrode holder comprises at least one of felt, cotton, chamois, and gauze.

13. The neurofeedback device according to claim 1, wherein the electrode holder may be releasably fastened to a plurality of locations on the electrode positioning device.

14. The neurofeedback device according to claim 1, wherein the electrode positioning device comprises a plurality of straps.

15. The neurofeedback device according to claim 14, wherein at least one electrode holder is releasably attachable to each strap.

16. The neurofeedback device according to claim 1, wherein the electrode positioning device is configured to position a plurality of electrodes only on a specific region of a patient's head.

17. A neurofeedback device, comprising:
a headband;
a cross strap having first and second ends, wherein at least one of the first and second ends is releasably attachable to the headband; and
a plurality of electrode holding elements releasably attachable to the cross strap and/or headband, wherein the placement of the plurality of electrode holding elements on the neurofeedback device is configured to be continuously adjustable.
